(19) United States
(12) Patent Application Publication
(10) Pub. No.: US 2010/0152563 A1
(43) Pub. Date: Jun. 17, 2010

(54) DUAL FLOW BLOOD MONITORING SYSTEM

(75) Inventors: Stephen Brian Turner, Cheltenham (GB); Andrew Ian Hart, Coleford (GB); James Whitt Weaver, Columbia, SC (US)

Correspondence Address: ADAMS INTELLECTUAL PROPERTY LAW, Suite 2350 Charlotte Plaza, 201 South College Street, CHARLOTTE, NC, 28244, US

(73) Assignee: SPECTRUM MEDICAL LIMITED, Gloucester (GB)

(21) Appl. No.: 12/620,968

(22) Filed: Nov. 18, 2009

Related U.S. Application Data
(60) Provisional application No. 61/116,148, filed on Nov. 19, 2008.
(60) Provisional application No. 61/119,049, filed on Dec. 2, 2008.

(51) Int. Cl. A61B 5/145; A61B 8/06

(52) U.S. Cl. 600/364; 600/454

(57) ABSTRACT

A dual blood flow monitoring system includes an electronic first sensor, an electronic second sensor, and an electronic monitoring device in communication with the first sensor and the second sensor. The first sensor monitors a first blood flow and the second sensor monitors a second blood flow. The electronic monitoring device is operative to receive signals from the sensors and to calculate and display a differential value representing a difference between the at least one parameter of the first blood flow and the at least one parameter of the second blood flow. A dual blood flow monitoring system is provided in a method for simultaneously monitoring extracorporeal arterial and venous blood flows, in which the sensors are placed in communication with respective tubing lines carrying the arterial and venous blood flows.

### CROSS REFERENCE TO RELATED APPLICATIONS

This non-provisional patent application claims the benefit of the priority and incorporates the contents of provisional patent application No. 61/119,049, entitled “Dual Flow Cardiac Monitoring System,” filed Dec. 2, 2008. This non-provisional patent application furthermore claims the benefit of the priority and incorporates the contents of provisional patent application No. 61/116,148, entitled “Template for Cardiac Monitoring System,” filed Nov. 19, 2008.

### TECHNICAL FIELD OF THE INVENTION

This invention relates generally to blood monitoring, and more particularly to a dual flow blood monitoring system.

### BACKGROUND OF THE INVENTION

Conventional arrangements for monitoring blood flow rates within flow channels typically rely upon ultrasonic flow sensors. Intraoperative flow measurements are used to monitor blood flow conditions in various flow channels during vascular, cardiac, transplant, plastic and reconstructive surgeries. Extracorporeal blood flow measurements are made externally of the patient during procedures in which the patient's blood is routed through a system such as a heart lung machine during a heart bypass operation. Blood flow is typically measured as blood passes through a sterile channel such as tubing. A typical flow sensor measures the direction and flow rate of extracorporeal blood flow in tubing by employing ultrasonic transit-time principles of operation.

An ultrasonic sensor can also be used to detect subtle changes in fluid density. These changes represent incidents known as emboli events. Because air and solid materials have different densities than that of fluid blood, it is possible for emboli events to be detected by sensing systems. When an emboli event occurs, a monitor informs the user that an incident has occurred and useful data representing that event is measured and recorded.

Conventional systems do not, however, provide any direct way to monitor and compare the arterial and venous blood flows respectively entering the patient through an arterial line and returning from the patient through a venous line. Accordingly, a need exists for a system that is capable of informing the clinician about flow discrepancies between these two channels.

### BRIEF SUMMARY OF THE INVENTION

Accordingly, there is a need for systems and methods for simultaneously monitoring arterial and venous blood flows during procedures that involve extracorporeal blood flow. According to one embodiment of the invention, a dual blood flow monitoring system includes an electronic first sensor, an electronic second sensor, and an electronic monitoring device in communication with the first sensor and the second sensor. The first sensor monitors a first blood flow and is operative to generate a first signal conveying information regarding at least one parameter of the first blood flow. The second sensor monitors a second blood flow and is operative to generate a second signal conveying information regarding at least one parameter of the second blood flow. The electronic monitoring device is operative to receive the first and second signals and to calculate and display a differential value representing a difference between the at least one parameter of the first blood flow and the at least one parameter of the second blood flow.

According to another embodiment of the invention, a method for simultaneously monitoring extracorporeal arterial and venous blood flows includes providing arterial blood to a patient through a first tubing line, receiving venous blood from the patient through a second tubing line, and providing a dual blood flow monitoring system that includes an electronic first sensor, an electronic second sensor, and an electronic monitoring device in communication with the first sensor and the second sensor, the electronic monitoring device having a display. The first sensor is maintained in communication with the first tubing line as the first sensor generates a first signal conveying information regarding the flow rate of the arterial blood. The second sensor is maintained in communication with the second tubing line as the second sensor generates a second signal conveying information regarding the flow rate of the venous blood. The flow rate of the arterial blood is determined based on the first signal, and the flow rate of the venous blood is determined based on the second signal. A differential value representing the difference between the flow rates of the arterial blood and the venous blood is calculated and is displayed by the monitoring device.

### BRIEF DESCRIPTION OF THE DRAWINGS

The subject matter that is regarded as the invention may be best understood by reference to the following description taken in conjunction with the accompanying drawing figures in which:

FIG. 1 is an environmental view of a dual blood flow monitoring system according to the invention, used in conjunction with a blood perfusion management system;

FIG. 2 is the dual blood flow monitoring system of FIG. 1 shown with an exemplary display of data;

FIG. 3 is a diagrammatic representation of a method, according to another embodiment of the invention, for determining whether blood flow is within specified limits and for detecting an emboli event;

FIG. 4 is a diagrammatic representation of a method, according to yet another embodiment of the invention, for determining and displaying differential flow rate, and for determining whether the differential flow rate exceeds a specified critical upper limit; and

FIG. 5 is the dual blood flow monitoring system of FIG. 1 shown with an exemplary display of data including scrolling time graph plots of blood flow data.

### DETAILED DESCRIPTION OF THE INVENTION

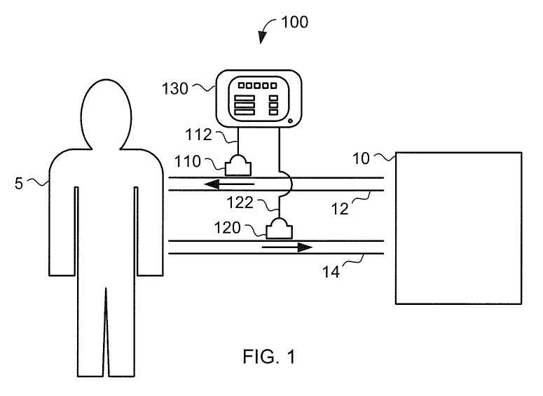

A typical perfusion management system 10 for treating the blood of a patient 5 is represented in FIG. 1 as used in conjunction with the dual blood flow monitoring system 100 according to at least one embodiment of the invention. The perfusion management system 10 represents any blood treatment system and in the illustrated example represents an artificial life support system that pumps and oxygenates the blood of a patient who is undergoing a procedure such as open heart surgery.

The perfusion management system 10 includes an arterial blood flow line 12, a venous blood flow line 14, and a blood treatment apparatus such as a heart-lung machine. Thus, in the illustrated example, the arterial blood flow line 12 provides oxygenated blood to the patient and the venous blood flow line 14 receives de-oxygenated blood from the patient. The perfusion management system 10 therefore represents a blood flow circuit for which the blood flows in the arterial line 12 and venous line 14 must be monitored at least in order to assure blood flow balance. Discrepancies in the flow entering the patient (arterial flow) and the flow returning from the patient (venous flow) can be caused by a surgeon's manipulation of the heart, system leaks, large bleeds, restriction of arteries or veins, and other losses resulting in poor perfusion and potentially the ingress of air due to reservoir drainage.

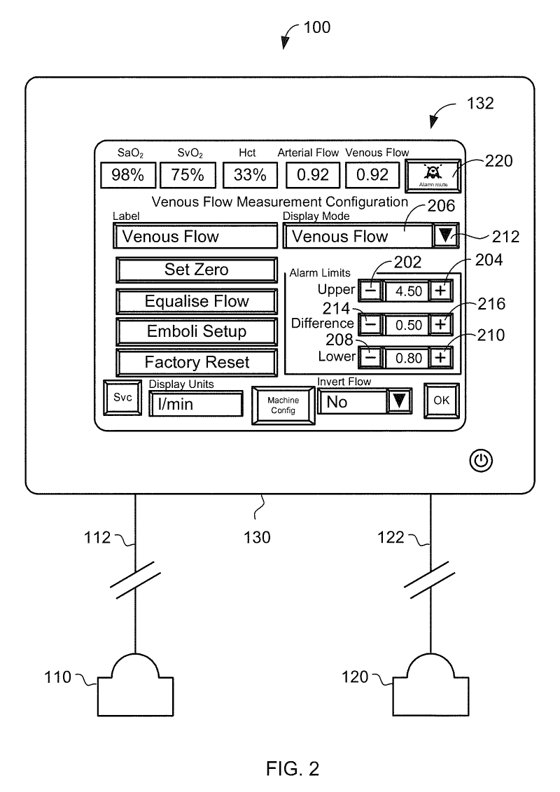

According to at least one embodiment of the invention, the dual blood flow monitoring system 100 includes an arterial sensor 110, a venous sensor 120, and a monitoring device 130. The system 100 is shown engaged in a monitored procedure in FIG. 1 and is shown separately in FIG. 2 with an exemplary display of measurement data. The arterial and venous sensors communicate electronically with the monitoring device 130 through respective communication lines 112 and 122 illustrated as cables although wireless communications are within the scope of these descriptions as well.

The sensors 110 and 120 represent a variety of sensor types including ultrasonic sensors. Exemplary sensor types include devices that measure oxygen saturation (SaO₂), mixed venous oxygen saturation (SvO₂), hematocrit (Hct) and hemoglobin concentration (Hb), volume flow rate, and other blood flow parameters. The sensors 110 and 120 provide their respective data signals to the monitoring device 130 along the communication lines 112 and 122 to facilitate both independent arterial and venous flow monitoring and differential calculations. Thus, real time measurements of blood flow parameters of both the arterial blood flow line 12 and the venous blood flow line 14 are achieved. This facilitates the measurement of differential blood flow rate, which becomes essential where clinicians are observing clinical parameters in real time.

The monitoring device 130 has the capability of analyzing signals along two independent channels, corresponding for example to the arterial and venous flow lines 12 and 14, for both flow and emboli events. Each measurement channel be assigned to a variety of blood flow parameters as the monitoring system can be connected to a variety of sensors and is able to display measured parameters on a numeric digital display such that represented for example in FIG. 2, and on a scrolling graph. The user can set the monitor to display absolute flow for both channels or actual flow from one channel and then the difference in flow between both two channels. If the difference in flow option is selected then one of the numeric digital displays will indicate the difference measurement but the scrolling graph will still show both actual flows as this provides the most information to the user. The monitoring device 130 includes a touch screen monitor 132, which defines a graphical user interface (GUI) that enables a user to navigate functionalities and control the device. The monitoring device 130 houses a processor which executes programming. The user controls the monitoring device 130 by touching user input buttons of the touch screen monitor 132. The monitoring device 130 performs all needed calculations, and generates data for the parameter values displayed by the monitor 132.

The arterial sensor 110 and the venous sensor 120 are each capable of generating signals from which respective flow rates and respective changes in density of the interrogated blood flows can be detected. By providing two sensors, the signals of which can be separately analyzed, the monitoring system 100 has two flow and emboli detection channels, and thus performs both absolute measurements of arterial and venous blood flow parameters and differential measurements regarding the differences between the blood flow parameters at the arterial and venous sensor placement locations. A rapid sampling speed permits flow and emboli detections at a rate of 1000 events per second, which is increased compared with previously existing monitoring systems. This makes the detection of an incident of air entering the blood flow more reliable due to the fact that such an incident represents an emboli event that can be detected multiple times as it passes a single sensor. The number of events detected will be displayed to the user along with a prediction of emboli ingress rate.

As the monitoring system 100 has the ability to detect the presence of emboli events on two channels, corresponding to the arterial and venous lines for example, it provides a significant advantage in that the presence of emboli, in particular air, present in the blood entering the body of a patient can be detected. A clinician typically previously relied upon an in-line arterial filter, which if triggered will shut down a pump, as a gross indicator.

In the event that surgeon manipulation or the application of vacuum to the returning venous line, intended to improve drainage, causes an emboli event, the clinician is notified. A venous emboli event can become an arterial emboli event. As the monitoring system 100 has the ability to detect the presence of emboli on two channels independently and differentially, the clinician is informed of nature and extent of the incident, and takes appropriate action. For example, the heart may be rested or the level of vacuum may be reduced.

An area of serious clinical interest is “cognitive deficit” brain dysfunction arising from open heart surgery and the ingress of air emboli in to the patient's brain. The monitoring system 100 provides quality information which includes the estimation of total air ingress. This feature may be used to study long term cognitive outcomes.

In at least one embodiment, the monitoring device 130 displays relevant patient data and provides navigation of the patient data through the touch screen monitor 132 for convenient use during patient-care procedures. For example, the monitoring system 100 may incorporate features described in the contents of provisional patent application No. 61/116,148, filed Nov. 19, 2008, and entitled “Template for Cardiac Monitoring System.”

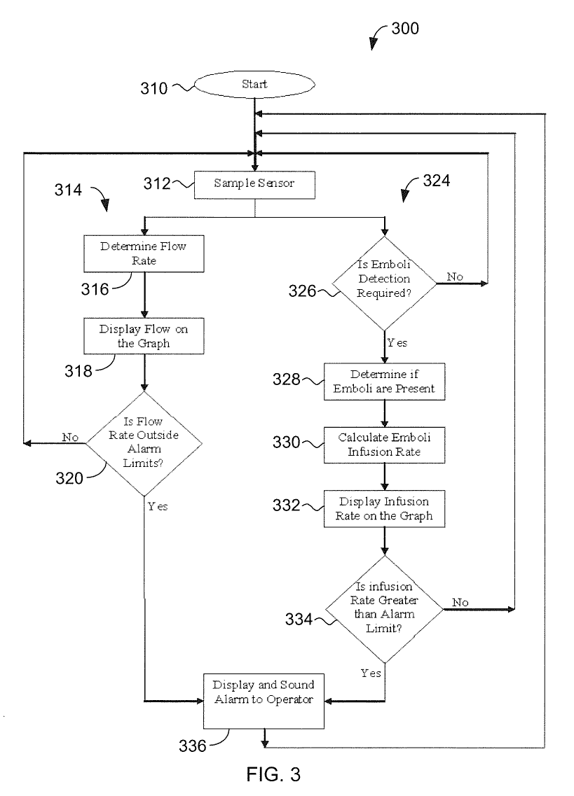

In at least one embodiment of the invention, a method for determining whether blood flow is within specified limits and for detecting an emboli event is provided. As shown in FIG. 3, a method 300 begins at a first step 310 representing the beginning of a procedure for which emboli detection is to be provided. For example, open heart surgery or another procedure represented in FIG. 1 may be initiated and the blood circulation of the patient 5 is placed under the care of the perfusion management system 10. In step 312 a sensor generates a data signal and that signal is sampled to determine blood flow rate along a sequence 314 of steps 316-320 and to detect emboli events along a parallel sequence 324 of steps 326-334. In step 312, the sensor may be for example the arterial sensor 110 or the venous sensor 120 as represented in FIG. 1. In either of the sequences 314 and 324, if a measurement is determined to fall outside of one or more specified limits, then an alert indication is displayed and an alarm is sounded at step 336.

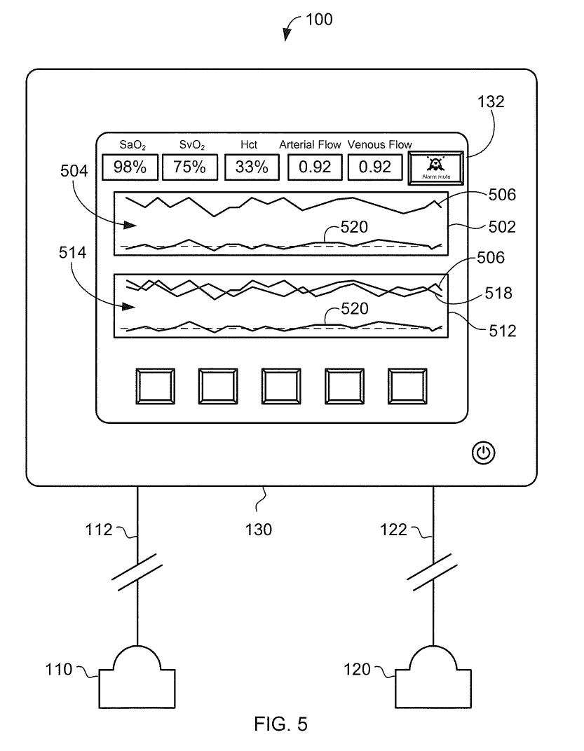

Regarding the sequence 314 for determining whether blood flow is within specified limits, at step 316 flow rate is determined from the data signal generated in step 312. This flow rate may represent arterial or venous blood flow rate. In step 318 the determined flow rate is displayed on a graph. For example, a numeric value and a scrolling time graph plotting flow rate as a time-varying value may be displayed on the monitoring device 130 as shown in the exemplary display of FIG. 5. A scrolling time graph 504 shows a plot 506 of a blood flow rate as a time-varying value in pane 502.

In step 320 the flow rate determined in step 318 is compared to upper and/or lower alarm limits. If the rate is determined as “Yes” to fall outside of alarm limits, then in step 336 an alert indication is displayed and an alarm is sounded. For example, an alert indicator may flash or be conspicuously displayed on the monitoring device 130 of FIG. 1 as an alarm sound is emitted. If the rate is determined as “No” regarding falling outside of alarm limits, indicating the flow rate is determined to fall within an acceptable range, then further data sampling is conducted in step 312. Thus the flow rate determining sequence 314 is part of an ongoing looping process for real time determination of flow rate as a time-varying value.

Regarding the sequence 324 for detecting emboli events, following the step 312 at which the sensor generates a data signal and that signal is sampled, a determination is next made as to whether emboli detection is required in step 326. If this determination results in “No,” then further data sampling is conducted in step 312 without execution of the full sequence 324. If this determination results in “Yes,” then further execution of the sequence 324 continues with step 328. In at least one embodiment of the monitoring system 100, emboli event detection can be disabled and enabled, such as by toggling a virtual button or otherwise setting a selection using the monitoring device 130 of FIG. 1 or other user interface means.

If emboli event detection is enabled, the presence of emboli is determined in step 328 by analyzing the data signal generated in step 312 by the sensor. For example, subtle changes in the fluid density in the blood flow can be detected by analyzing the data signal generated in step 312. Once an emboli event is determined to be present, the method 300 continues with step 330 where the emboli infusion rate is calculated. In step 332 the calculated emboli infusion rate is displayed. For example, a numeric value and a scrolling time graph plotting infusion rate as a time-varying value may be displayed on the monitoring device 130 of FIG. 1.

In step 334 the emboli infusion rate calculated in step 330 is compared to an upper alarm limit. If the rate is determined as “Yes” to be greater than an upper limit, then in step 336 an alert indication is displayed and an alarm is sounded. For example, an alert indicator may flash or be conspicuously displayed on the monitoring device 130 of FIG. 1 as an alarm sound is emitted. If the calculated emboli infusion rate is determined as “No” with regard to exceeding the upper alarm limit, indicating the emboli infusion rate is determined to fall below a predetermined or user selected value, then further data sampling is conducted in step 312. Thus the emboli event detection sequence 324 is part of an ongoing looping process for real time detection of emboli events.

In at least one embodiment of the monitoring device 130, the upper and lower alarm limits against which the determined flow rate is compared in step 320 is adjustable by the user of the device. For example, in FIG. 2, which provides an exemplary display of the touch screen monitor 132, by pressing the virtual buttons 202 and 204 a user prompts the monitoring device 130 to respectively decrease and increase the upper alarm limit. In the illustrated example, the display relates to the venous flow and adjustments by way of the virtual buttons 202 and 204 relate to venous flow alarm limits as indicated for example in pane 206. The lower alarm limits for the venous flow are adjusted by similar use of the buttons 208 and 210, which respectively lower and raise the lower alarm limits. The pane 206 additionally provides a drop down menu button 212 to permit the user to navigate to arterial flow adjustment buttons as well. The upper alarm limit against which the calculated emboli infusion rate is compared in step 334 can be adjusted by the user as well, for example by navigating to an emboli setup menu and by pressing adjustment buttons.

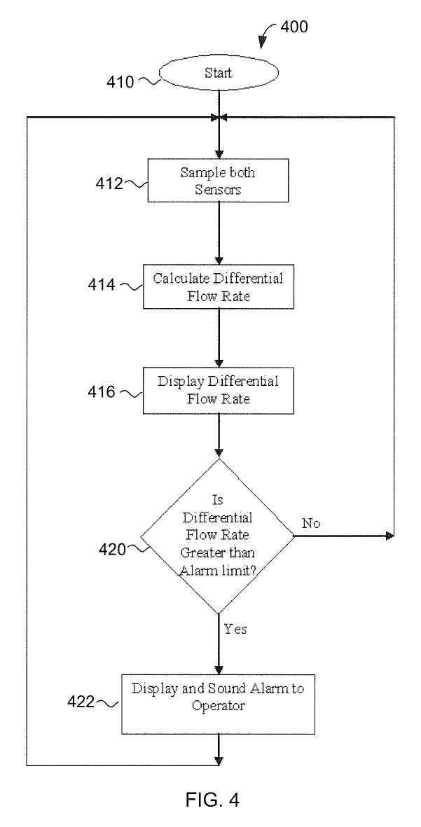

Furthermore, in at least one other embodiment of the invention, a method 400 as represented in FIG. 4 is provided for determining and displaying differential flow rate, and for determining whether the differential flow rate exceeds a specified critical upper limit. The differential flow rate is defined as the difference between arterial and venous flows. Thus, when the differential flow rate differs significantly from zero, an imbalance between blood taken from a patient and blood provided to a patient is occurring. As any significant such imbalance is typically undesirable or at least deserves attention and care by a clinician, the difference defined herein may be calculated as an unsigned absolute value or as a signed value indicative of which of the arterial and venous blow flow rates exceeds the other.

As shown in FIG. 4, a method 400 begins at a first step 410 representing the beginning of a procedure for which differential flow rate determination is to be provided. For example, open heart surgery or another procedure represented in FIG. 1 may be initiated and the blood circulation of the patient 5 is placed under the care of the perfusion management system 10. In step 412 of FIG. 4, the arterial sensor 110 and the venous sensor 120 generate respective data signals that are sampled. In step 414, the differential flow rate is calculated by comparing the arterial and venous blood flow rates.

In step 416, the differential flow rate is displayed. For example, a numeric value and a scrolling time graph plotting differential flow rate as a time-varying value may be displayed on the monitoring device 130. In at least one embodiment of the method 400, the arterial flow rate, the venous flow rate, and the differential flow rate are all displayed to provide the most information to the user. For example, in the scrolling time graph 514 of pane 512 of FIG. 5, a plot 506 represents arterial flow rate, a plot 518 represents the venous flow rate, and a plot 520 represents the differential flow rate. In pane 502, however, only the arterial flow rate plot 506 and the differential flow rate plot 520 are displayed to provide a simpler presentation of measured blood flow parameters.

In step 420 the differential flow rate calculated in step 416 is compared to an alarm limit. If the rate is determined as “Yes” to be greater than an upper limit, then in step 422 an alert indication is displayed and an alarm is sounded. For example, an alert indicator may flash or be conspicuously displayed on the monitoring device 130 of FIG. 1 as an alarm sound is emitted. If the calculated differential flow rate is determined in step 420 as “No” with regard to exceeding the upper limit, indicating that the arterial and venous blood flow rates are sufficiently comparable, then further data sampling is conducted in step 412. Thus the method 400 for determining and displaying differential flow rate and for determining whether the differential flow rate exceeds a specified critical upper limit is conducted as an ongoing looping process in real time.

In at least one embodiment of the monitoring device 130, the alarm limit against which the differential flow rate is compared in step 420 is adjustable by the user of the device. For example, in FIG. 2, by pressing the virtual buttons 214 and 216 a user prompts the monitoring device 130 to respectively decrease and increase the differential flow rate alarm limit.

Alert indications and visible alarms described herein may be displayed in various fashions. For example, in at least one embodiment of the invention, an alert indicator pane 220 is present in the top right area of the display of the monitoring device 130 as shown in FIG. 2. When an alert condition has been reached, the alert indicator pane 220 blinks while a beeping audible alarm sounds. The pane 220 also serves as a mute button by which the audible alarm is silenced by a user.

The monitoring device 130 includes a processor that performs calculations necessary for the methods and functions described above based on computer-readable program instructions (software) utilized by an operating system. The processor, software, and additional drivers facilitate functionalities including data display and user inputs. The system further includes memory, optionally both read only memory (ROM) and random access memory (RAM). The memory is used to store a basic input/output system, facilitate routines that transfer data between elements within the system.

The monitoring device 130 in at least one embodiment includes at least one storage device, such as a hard disk drive, CD/DVD Rom drive or optical disk drive for storing information on various computer-readable media, such as a hard disk, removable magnetic disk, CD/DVD-ROM disk or flash memory. The storage device facilitates that patient data may both be provided to the monitoring device 130 for display and analysis and provided by the monitoring device 130 for use or storage in other systems. The monitoring device 130 may additionally provide wireless connectivity for exchanging data with other devices, a central server, or the interne where patient information and software updates are provided.

In at least one embodiment, the monitoring device 130 provides wireless as well as RS-232, USB, I.R., and SD memory card connectivities to facilitate data storage, post-operative analysis and connections to external electronic charting systems.

While specific embodiments of the present invention have been described, it will be apparent to those skilled in the art that various modifications thereto can be made without departing from the spirit and scope of the invention. Accordingly, the foregoing description of the preferred embodiment of the invention and the best mode for practicing the invention are provided for the purpose of illustration only and not for the purpose of limitation.

What is claimed is:

1. A dual blood flow monitoring system comprising:
an electronic first sensor for monitoring a first blood flow, the first sensor operative to generate a first signal conveying information regarding at least one parameter of the first blood flow;
an electronic second sensor for monitoring a second blood flow, the second sensor operative to generate a second signal conveying information regarding at least one parameter of the second blood flow; and
an electronic monitoring device in communication with the first sensor and the second sensor, the electronic monitoring device operative to receive the first and second signals, and to calculate and display a differential value representing a difference between the at least one parameter of the first blood flow and the at least one parameter of the second blood flow.

2. A dual blood monitoring system according to claim 1, wherein:
the first sensor comprises an ultrasonic sensor operative to generate the first signal conveying information regarding the rate of the first blood flow in volume per time;
the second sensor comprises an ultrasonic sensor operative to generate the second signal conveying information regarding the rate of the second blood flow in volume per time; and
the monitoring device is operative to calculate and display the differential value representing the difference between the rate of the first blood flow and the rate of the second blood flow.

3. A dual blood monitoring system according to claim 2, wherein the monitoring device is operative to generate an audible or visible alarm signal if the rate of the first blood flow exceeds an upper limit or falls below a lower limit.

4. A dual blood monitoring system according to claim 3, wherein the electronic monitoring device comprises a user input device and is operative to adjust the upper and lower limits according to user inputs.

5. A dual blood monitoring system according to claim 1, further comprising:
a first cable connected to the first sensor and to the monitoring device, the first cable capable of conveying the first signal from the first sensor to the monitoring device; and
a second cable connected to the second sensor and to the monitoring device, the second cable capable of conveying the second signal from the second sensor to the monitoring device.

6. A dual blood monitoring system according to claim 1, wherein the electronic monitoring device is operative to generate an audible or visible alarm signal if the differential value exceeds a threshold value.

7. A dual blood monitoring system according to claim 6, wherein the electronic monitoring device comprises a user input device and is operative to adjust the threshold value upon receipt of user input.

8. A dual blood monitoring system according to claim 7, wherein the user input device comprises a touch screen monitor operative to display at least the differential value and to receive user inputs.

9. A dual blood monitoring system according to claim 1, wherein the monitoring device is operative to display a scrolling time graph plotting time-varying values representing the at least one parameter of the first blood flow and the at least one parameter of the second blood flow.

10. A dual blood monitoring system according to claim 1, wherein the monitoring device is operative to display a scrolling time graph plotting time-varying values representing the at least one parameter of the first blood flow and the differential value.

11. A dual blood monitoring system according to claim 1, wherein:
the first sensor comprises an ultrasonic sensor operative to generate the first signal conveying information regarding the density of the first blood flow;
the second sensor comprises an ultrasonic sensor operative to generate the second signal conveying information regarding the density of the second blood flow; and
the monitoring device is operative to calculate and display the emboli infusion rate of the first blood flow based on the first signal, and to calculate and display the emboli infusion rate of the second blood flow based on the second signal.

12. A dual blood monitoring system according to claim 1, wherein the first sensor comprises an ultrasonic sensor operative to generate the first signal conveying information regarding the oxygenation of the first blood flow.

13. A method for simultaneously monitoring extracorporeal arterial and venous blood flows, the method comprising:
providing arterial blood to a patient through a first tubing line;
receiving venous blood from the patient through a second tubing line;
providing a dual blood flow monitoring system that includes an electronic first sensor, an electronic second sensor, and an electronic monitoring device in communication with the first sensor and the second sensor, the electronic monitoring device having a display;
maintaining the first sensor in communication with the first tubing line as the first sensor generates a first signal conveying information regarding the flow rate of the arterial blood;
maintaining the second sensor in communication with the second tubing line as the second sensor generates a second signal conveying information regarding the flow rate of the venous blood;
determining the flow rate of the arterial blood based on the first signal;
determining the flow rate of the venous blood based on the second signal;
calculating a differential value representing the difference between the flow rates of the arterial blood and the venous blood; and
displaying the differential value on the display of the monitoring device.

14. A method according to claim 13, further comprising displaying the flow rates of the arterial blood and the venous blood with the differential value.

15. A method according to claim 13, wherein displaying the flow rates of the arterial blood and the venous blood comprises displaying a scrolling time graph plotting time-varying values representing the flow rates of the arterial blood and the venous blood.

16. A method according to claim 13, further comprising generating an audible or visible alarm signal if the differential value exceeds a threshold value.

17. A method according to claim 16, further comprising receiving a user input and adjusting the threshold value according to the user input.

18. A method according to claim 13, further comprising generating an audible or visible alarm signal if the flow rate of the arterial blood or the flow rate of the venous blood exceeds an upper limit or falls below a lower limit.

19. A method according to claim 18, further comprising receiving a user input and adjusting the upper limit or the lower limit according to the user input.

20. A method according to claim 13, further comprising calculating and displaying the emboli infusion rate of the arterial blood based on the first signal, and calculating and displaying the emboli infusion rate of the venous blood based on the second signal.
